(19) United States
(12) Patent Application Publication
(10) Pub. No.: US 2013/0298063 A1
(43) Pub. Date: Nov. 7, 2013

(54) Active Overlay of Diabetes Management Information on a Display

(71) Applicant: MEDTRONIC MINIMED, INC., Northridge, CA (US)

(72) Inventors: Kelly Joy, La Canada, CA (US); Theodora Padron, Sylmar, CA (US)

(73) Assignee: MEDTRONIC MINIMED, INC., Northridge, CA (US)

(21) Appl. No.: 13/874,661

(22) Filed: May 1, 2013

Related U.S. Application Data
(60) Provisional application No. 61/643,041, filed on May 4, 2012.

(51) Int. Cl. G06F 19/00; A61M 5/172; A61B 5/145

(52) U.S. Cl. CPC G06F 19/3406; A61B 5/14532; A61M 5/172
USPC 715/771; 600/365; 604/246

(57) ABSTRACT

The present invention describes an active overlay of real-time glucose and/or insulin information on a display for convenient monitoring and control of information for the management of diabetes. The superimposed images on display can be varied based on automated and/or programmable processes and/or alerts of the system. In various embodiments, the system can include a console and/or remote control to allow the user to control the overlay display. In specific embodiments, the system can communicate with a glucose sensor and/or insulin infusion device.

### CROSS-REFERENCE TO RELATED APPLICATIONS

This application claims priority to U.S. Provisional Patent Application Ser. No. 61/643,041, filed on May 4, 2012, which is incorporated by reference in its entirety.

### FIELD OF THE INVENTION

This invention relates to an overlay of diabetes management information on a display, and in particular embodiments, to an active overlay of real-time glucose, glucose sensor, insulin infusion device, and/or insulin information on a display that includes automated, pre-programmed, remotely controlled, and/or alarm capabilities.

### BACKGROUND OF THE INVENTION

Individuals having Type 1 and many individuals having Type 2 diabetes use insulin daily to control their blood glucose (BG) levels. To deliver the insulin to the body, the diabetic patients use insulin delivery devices, including external infusion pumps or patches, injection pens, and implantable delivery systems. Throughout the day, the diabetic patient measures his or her BG level using a BG measurement device, such as a test strip meter, to determine if treatment is needed, be it with glucose to raise glucose levels or insulin to lower glucose levels. In addition, the diabetic patient may use a continuous glucose measurement system, to monitor sensor glucose (SG) throughout the day. The glucose measurement devices can use different methods to measure a patient's glucose level, including testing a sample of the patient's blood to determine BG or using a sensor in contact with interstitial fluid to determine SG. The testing and monitoring of an insulin-dependent individual's BG level is performed several times throughout the day and can inconvenience his or her daily routine.

One drawback of current methods of monitoring a patient's glucose and insulin data is the inability to seamlessly incorporate a personalized diabetes management system into the patient's daily routine. Currently, televisions, smart phones, electronic devices having displays and devices having graphical user interfaces are a part of everyday life for many people. As such, many individuals are familiar with the features and controls of these devices. However, the devices do not currently provide an active overlay on the device display that provides real-time diabetes management information. Another disadvantage of current devices' displays is the inability to provide an alert to warn the user of a medical condition which requires the user's attention. Current devices cannot augment the device display or otherwise provide an alert from the device to the user of the medical condition. Current devices also do not allow for interactive control of diabetes devices such as insulin infusion devices.

### BRIEF SUMMARY OF THE INVENTION

Embodiments of the present invention provide overlays, and systems and methods using the overlays for convenient monitoring and management of diabetes for diabetic patients, which obviate for practical purposes, the above mentioned limitations.

According to an embodiment of the invention, an overlay and/or system for displaying diabetes management information on a display can include a display screen, an underlying display content on the display screen, and at least one diabetes data value. At least one diabetes data value is superimposed over the underlying display content on the display screen, thereby forming an overlay on the display.

In other embodiments, an overlay signal can include at least one diabetes data value and the system can include a processor for transmitting the overlay signal to the display screen, the processor thereby generating an overlay including the at least one diabetes data value superimposed over the underlying display content on the display screen.

In some embodiments, at least one diabetes data value may be continuous and includes at least one real-time data value. In exemplary embodiments, the diabetes data value can be one or more glucose data value and/or insulin data value. The glucose data value can include, e.g., at least one of the following data values: a blood glucose level, a predicted blood glucose level, a plurality of blood glucose levels over a specified period of time, a glucose sensor data value such as a glucose sensor power supply indicator, or a combination of any of the above-listed data values. The insulin data value can include, e.g., at least one of: an insulin infusion device power supply indicator, an insulin basal rate, an insulin bolus, an insulin reservoir supply indicator, or a combination of one or more of any of the above insulin data values.

In some embodiments, the overlay can include an analyte reading. Other embodiments of the overlay may include a numeric value, a text, a chart, a graph, an image, a sound, or a combination of one or more of the above. As a non-limiting example, the overlay image may include one or more arrows pointing up to indicate an increased blood glucose level. As another example, the overlay image may include one or more arrows pointing down to indicate a decreased blood glucose level. In certain embodiments, the overlay text can include at least one flashing message, scrolling message, or both.

In embodiments having a processor, the processor may analyze the underlying display content and adjust the overlay in relation to the underlying display content. For example, the processor may include a screensaver function to display the overlay when the underlying display content is inactive.

Further embodiments of the system include a memory having an alert criterion range corresponding to one or more stored diabetes data values. In such embodiments, the processor can compare at least one diabetes data value received with the alert criterion range, generate an alert upon receiving at least one diabetes data value within the alert criterion range, and provide an alert signal including the alert to the display screen.

In specific embodiments, the alert can provide at least one of the following: change the size of the overlay in relation to the underlying display content, expand the diabetes management information in the overlay, provide an alert sound, provide an alert message, provide an alert image, freeze the underlying display content, change a color of the overlay, change a color of the underlying display content, or any combination of the above-listed items.

In further embodiments, the processor can end the alert upon receiving either a diabetes data value outside the alert criterion range, an acknowledgment signal, or both a diabetes data value outside the alert criterion range and an acknowledgment signal. In yet further embodiments, the processor can transmit an alert signal including the alert to a remote device adapted to receive and provide the alert.

In yet further embodiments, the system can include a remote control. In several embodiments, the processor can be adapted to receive and process instructions transmitted from the remote control. The overlay may include a programming screen, which may work in conjunction with the remote control. In some embodiments, the system may include a motion detector adapted to receive motion commands.

In additional embodiments, the display screen can be located on a first object and the processor can be located in the first object. In alternative embodiments, the display screen can be located on a first object and the processor can be located in an external device in communication with the first object. In specific embodiments, the external device can be a console.

In yet further embodiments, the system can work with a glucose sensor and glucose sensor transmitter, where the glucose sensor transmitter is capable of transmitting at least one glucose data value to the processor. In embodiments including a glucose sensor, the glucose sensor can be adapted to receive and process a first communication transmitted from a remote control via the processor.

In other embodiments, the system can work with an insulin infusion device and insulin infusion device transmitter, where the insulin infusion device transmitter is capable of transmitting at least one insulin data value to the processor. In some embodiments the system can include an insulin reservoir. In embodiments including an infusion device, the insulin infusion device can be adapted to receive and process a second communication transmitted from the remote control via the processor.

Embodiments of methods for overlaying glucose information on a display are also describe herein. A non-limiting method can comprise the steps of: providing a display screen; receiving an input display signal for providing an underlying display content on the display screen; receiving an overlay signal including a diabetes data value, wherein the diabetes data value is at least one of a glucose data value, an insulin data value, a glucose sensor data value, and an insulin infusion device data value; providing a processor for generating an overlay based the at least one diabetes data value; and superimposing the overlay over the underlying display content on the display screen.

Other features and advantages of the invention will become apparent from the following detailed description, taken in conjunction with the accompanying drawings which illustrate, by way of example, various features of embodiments of the invention.

### BRIEF DESCRIPTION OF SEVERAL VIEWS OF THE DRAWINGS

A detailed description of embodiments of the invention will be made with reference to the accompanying drawings, wherein like numerals designate corresponding parts in the several figures.

FIG. 1 is a block diagram of an embodiment of a system for overlaying diabetes management information on a display in accordance with embodiments of the present invention;

FIG. 2 is a block diagram of an embodiment of a system for overlaying diabetes management information on a display in accordance with embodiments of the present invention;

FIG. 3 is a flowchart for executing an alert in accordance with an embodiment of the present invention;

FIG. 4 is a representation of an overlay on a display in accordance with an embodiment of the present invention;

FIG. 5 is a further representation of an overlay on a display in accordance with an embodiment of the present invention;

FIG. 6 is a still further representation of an overlay on a display in accordance with an embodiment of the present invention;

FIG. 7 is a yet further representation of an overlay on a display in accordance with an embodiment of the present invention;

FIG. 8 is an additional further representation of an overlay on a display in accordance with an embodiment of the present invention; and

FIG. 9 is an additional further representation of an overlay on a display in accordance with an embodiment of the present invention.

### DETAILED DESCRIPTION OF THE INVENTION

As shown in the drawings for purposes of illustration, the invention is embodied in a system for providing an overlay of diabetes management information superimposed on a display for improved management of diabetes. The system can utilize a device that is already used in a person's daily routine to display and control the diabetes management information without interfering with the regular function of the electronic device, unless the system is programmed or controlled to do so. In certain embodiments of the present invention, the overlay includes glucose data values, insulin data values, glucose sensor data values, insulin infusion device data values, or a combination of any of the above. In further embodiments, other medical information, such as cardiac information, vital signs, respiration, blood pressure or the like may be overlaid. The system can also include alerts or alarms to warn the user of certain conditions requiring further attention. The system allows the user to simultaneously view the overlay information and the background display.

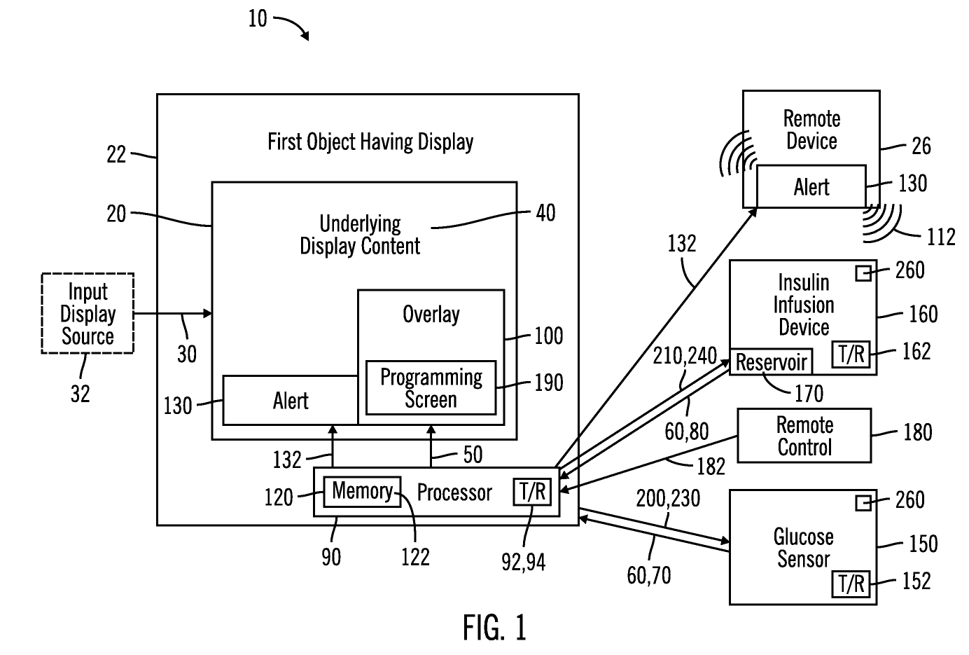

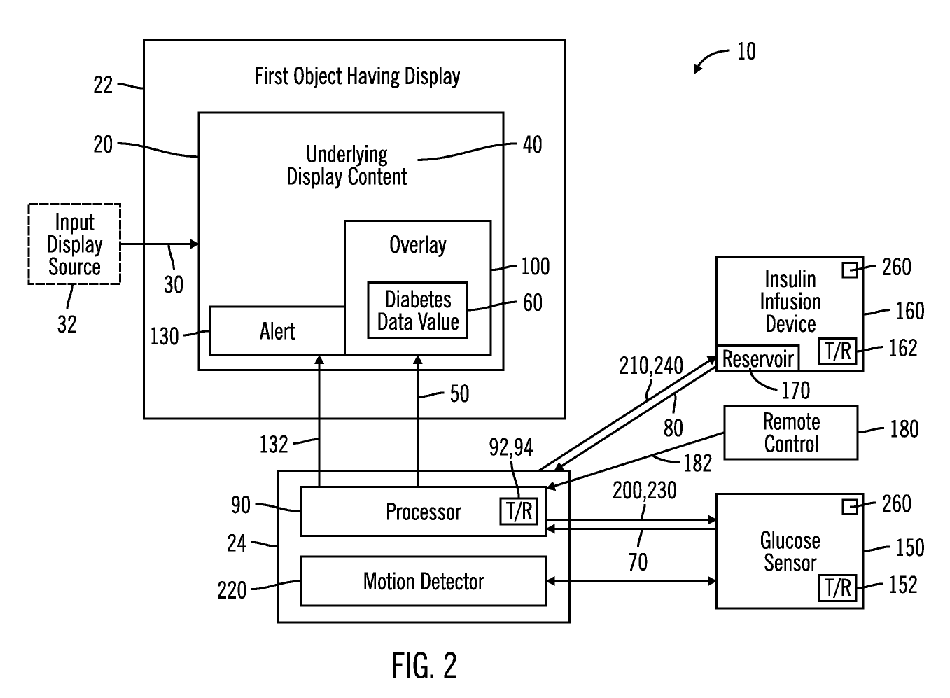

Generally shown in FIGS. 1 and 2, an embodiment of the system 10 for overlaying diabetes management information on a display includes an overlay 100 on a display screen 20 on a first object 22. The overlay 100 can be superimposed on a display screen 20 of any object, device or apparatus 22 including, but not limited to a television, smart phone, cell phone, personal data assistant, computer monitor, laptop, netbook, tablet, high functioning monitor, digital picture frame, refrigerator, display in a vehicle, or the like. The overlay information 100 is provided over an underlying display content 40, or background, on the display screen 20. The underlying display content 40 can be provided by an input display signal 30 from any source 32. As a non-limiting example, in embodiments where the display screen 20 is that of a television, the input display signal 30 providing the underlying display content 40 can be, e.g., an antenna, cable, Wi-Fi, satellite signal, or the like.

In particular embodiments, the overlay display content 100 can be provided by an overlay signal 50. The overlay signal 50 can comprise at least a portion of diabetes management information, i.e., at least one diabetes data value 60. The diabetes data value 60 can include any information relating to the management of diabetes, including glucose data values 70 and insulin data values 80, or information relating to the management and control of diabetes devices such as insulin infusion devices 160 or glucose sensors 150. The information can be displayed on the overlay 100 in the form of text 104, images 110, numeric values 102, charts 107, graphs 108, or any combination thereof. The diabetes data values 60 can be continuous and can include real-time actual data values and predicted future data values. The charts 107 and graphs 108 can show data value trends over a specified time period. The charts 107 and/or images 110 may include arrows 114 that show the direction of change that a data value trend is moving. The overlay can include multiple arrows 114 pointing up or down, and can also increase or decrease the number of arrows 114 on display in accordance with the trending of the data values 60. The text 104 provided in the overlay 100 can be in the form of a flashing message 105 or a scrolling message 106. The preceding examples of arrows 114 and flashing 105 or scrolling 106 text can also be set as alerts 130 in the system 10, as discussed further below. All or portions of the information in the overlay 100 and/or alerts 130 can flash or change color.

In some embodiments, sound 112 can accompany the overlays 100. The sound 112 can change on different overlays 100 to help the user distinguish a type of alert 130 or identify the information or data values being conveyed via the overlay 100. The user can select personalized sounds 112 from a list to customize the sounds 112 to the user's tastes. Possible variations of sounds 112 can include, but are not limited to imported MP3 files, a user's own vocal messages and/or vocal messages of the user's loved one.

In embodiments, the appearance, size, colors, sounds, alerts, and choice of information and data values of the overlay 100 can be user configurable. For example, the user may program and/or configure the overlay 100 from the device having the display screen 20, from a remote control 180, or from an external device 24 such as a console managing and sending the data values out to the display screen 20. Further details regarding the overlay information 100, overlay format, and alerts 130 are described below.

In particular embodiments, the system 10 further includes a processor 90 that can receive at least one diabetes data value 60 and/or the input display signal 30, generate the overlay 100 of data values and resulting information, and superimpose the overlay 100 over the underlying display content 40 on the display screen 20. In some embodiments, the processor can combine at least one diabetes data value 60 and the underlying display content 40 to generate an overlay 100 on the display 20. In some embodiments, the processor 90 can combine an overlay signal 50 having at least one diabetes data value 60 and the input display signal 30 to generate an overlay 100. The processor 90 can transmit the overlay 100 to the display screen 20 via an overlay signal 50. A processor 90 that is capable of generating the overlay 100 as described herein can include a computer chip, a hardware device, or can work with a widget, an application, or the like.

As shown generally in FIG. 1, embodiments of the processor 90 can be hardware installed in a first object 22 having the display screen 20. In other embodiments, shown generally in FIG. 2, the processor 90 can be a separate hardware device or a hardware device installed in an external device 24, such as a console, that is capable of communicating with the first object 22 having the display screen 20. The processor 90 in the console can transmit signals and/or information to the first object 22 and/or receive signals and/or information from the first object utilizing one or more transmitters and/or receivers.

As another non-limiting example, where the processor 90 works with a widget or application, the widget or application can be installed in the first object 22 having the display screen 20 or in an external device 24, for example, a console. The widget or application can retrieve diabetes data values for the overlay from a server in communication with an insulin infusion device 160 or a glucose sensor 150. In such embodiments, the server, insulin infusion device 150, and glucose sensor 160 can each include transmitters and/or receivers or input/output mechanisms to communicate with one another.

The processor 90 can include various manual, pre-programmed or automated functions. For example, the processor 90 can be capable of analyzing the underlying display content 40 and altering the overlay 100 or adjusting the overlay 100 in relation to the underlying display content 40. In this regard, when the system 10 is utilized with (e.g., a television) the processor 90 may be able to recognize commercials in the underlying display content 40 from the input display signal 30 and increase the size and/or information content of the overlay 100 in relation to the display background 40 for the duration of the detected commercial break. In another embodiment, the processor 90 can include a screensaver function to display the overlay 100 when the underlying display content 40 is inactive.

In embodiments where the processor 90 is not installed in an object 22 having a display screen 20 and can not only generate an overlay 100, but can also analyze the underlying display content 40 of the object 22 having the display screen 20, the processor 90 can include an input or receiver 94 for the input display signal 30, such as Ethernet, Wi-Fi, coaxial, HDMI, component, AV cables and the like. For example, the processor 90 could include this input 94 where it is not installed in the object 22 having the display screen 20 and is used to detect inactivity or commercials played on the underlying display content 40. The processor 90 can further include an output or transmitter 92 to the object 22 having the display screen 20 for embodiments where the processor 90 can alter the underlying display content 40 to the object 22 having the display screen 20, e.g., freeze the underlying display content 40 during an alert 130. The processor 90 can communicate with other components of the system via wired or wireless signal. Wireless technology can include Wi-Fi, Bluetooth, ZigBee, along with cellular communication standards such as but not limited to CDMA and GSM. Other communications include, but are not limited to IR and/or optical communication methods.

As illustrated in FIG. 1, in embodiments where a display screen 20 is located on a first object 22 or apparatus having a display screen 20, the processor 90 can also be located or incorporated in the first object 22. The processor 90 can be hardware or work with a program, widget or application installed in the first object 22. The processor 90 is capable of communication with the first object 22 and other devices that can be included in the system 10 such as a glucose sensor 150, insulin infusion device 160 and insulin reservoir 170.

In an alternative embodiment shown in FIG. 2, where the display screen 20 is located on a first object 22 or apparatus having a display screen 20, the processor 90 can be located or incorporated in an external device, apparatus or discrete console 24. The processor 90 and/or external device 24 can be in communication with the first object 22, or a widget or application connected with the first object 22.

As a non-limiting example, the external device 24 can be a console. The overlay 100 is generated by a processor 90 on the discrete console configured to communicate with an insulin pump 160 and/or a transmitter associated with a glucose sensor 150. The discrete console may be connected to the first object 22 having a display screen 20 using standard audio/visual coupling techniques such as, but not limited to HDMI, DisplayPort, wireless HDMI, Ethernet, Wi-Fi, and the like. In embodiments where the device having a display is a television defined to accept widgets or applications, the overlay 100 can be generated using a processor 90 that is a specialized application with access to real-time glucose values via a transmitter 92 and/or receiver 94. In still other embodiments, a transmitter 92 and/or receiver 94 is coupled to a third party game console, such as but not limited to a PlayStation®, Xbox®, Kinect®, Wii® or the like. In these instances, the discrete game console is coupled to a display 20 and either the processor 90 function is executed directly by the console, or a processor 90 is attached to the console to generate the overlay 100.

The overlay 100 of at least one diabetes data value 60 can include glucose data values 70, insulin data values 80, or a combination thereof. The glucose data values 70 can be derived from different methods including, but not limited to discrete tests from a meter, continuous glucose monitoring device, and measurements of glucose oxidase or optical sensors.

In particular embodiments, the one or more glucose data values 70 can include a blood glucose level 72, a predicted blood glucose level 74, a plurality of blood glucose levels over a specified period of time 76, or a combination thereof. The aforementioned glucose data values 70 can be received from a glucose sensor 150 and thus include sensor glucose levels. As described above, the information displayed on the overlay 100 can include text 104, images 110, numeric values 102, charts 107, graphs 108, or any combination thereof. The charts 107 and graphs 108 can show data value trends over a specified time period and arrows 114 can show the direction of change that a trend is moving. For example, the overlay image 110 can be an arrow 114 pointing up to indicate an increased blood glucose level 72 or a hyperglycemic condition. Conversely, the overlay image 110 can be an arrow 114 pointing down to indicate a decreased blood glucose level 72 or a hypoglycemic condition. The overlay can include multiple arrows 114 pointing up or down, and can also increase or decrease the number of arrows 114 on display in accordance with the trending of the data values 60. All or portions of the information in the overlay 100 and/or alerts 130 can flash or change color.

In further embodiments, the glucose data value 70 can also include other information relating to a glucose sensor 150, in addition to the glucose data values 70 described above. For example, the glucose data value 70 can include a glucose sensor power supply indicator 79 to indicate the power supply or battery life remaining in the glucose sensor 150. In some embodiments, the overlay 100 can include a calibration indicator 78 which indicates the time remaining until the glucose sensor 150 requires calibration. The glucose sensor 150 can have a glucose sensor transmitter 152 to send the glucose data values 70 to the processor 90. The processor 90 is adapted to receive the glucose data values 70 to comprise part of the overlay signal 50. The processor 90 can have one or more transmitters 92 and/or receivers 94 to transmit, receive, or both transmit and receive the signals and data of the system 10. In alternative embodiments, the sensor can also provide data values of analyte readings such as oxygen, pH, lactate, or the like.

In certain embodiments, an insulin infusion device 160 works with the system 10. The overlay 100 can provide information relating to the insulin infusion device 160 and the insulin infusion parameters. Accordingly, the insulin data value 80 can provide an insulin infusion device power supply indicator 82 to indicate the remaining power supply or battery life remaining in the device. The overlay 100 can also include information pertaining to an insulin reservoir 170 that can be included in the system 10, such as an insulin reservoir supply indicator 88 to indicate the amount of insulin supply remaining in an insulin reservoir 170. Other insulin data values 80 regarding insulin infusion parameters can include an insulin basal rate, an insulin bolus. In some embodiments, the insulin data values 80 can be shown on the overlay 100 as text 104 or numeric values 102. In other embodiments, charts 107 and/or graphs 108 can show insulin data value 80 trends over a specified time period. The charts 107 and/or numbers 102 may expand or change size over time depending on the information being shown. The overlay 100 could be interleaved to be a rotating graph 108 and/or included into one of the existing graphs 108. The overlay 100 can comprise a combination of any of the aforementioned features.

In various embodiments, like the glucose sensor 150, the insulin infusion device 160 can have an insulin infusion device transmitter 162 to send the insulin data values 80 to the processor 90. The processor 90 is adapted to receive the insulin data values 80 as part of the overlay signal 50. The processor 90 can have a receiver 94 to receive the signals and data. The insulin infusion device 160, insulin reservoir 170 and glucose sensor 150 may be combined as one component of the system 10 or exist as separate components of the system 10. In embodiments where the glucose sensor 150 and insulin infusion device 160 are combined as one component of the system 10, either the glucose sensor transmitter 152, the insulin infusion device transmitter 162, or another transmitter can send both glucose data values 70 and insulin data values 80 to the processor 90. Some embodiments of the invention may utilize a relay system comprising one or more relay transmitters and receivers to provide communication between the components of the system.

Additional diabetes data values 60 provided in the overlay 100 can include information related to closed loop performance. For instance, how the algorithm is tracking or adapting for the user. It could show programmed parameters, what is expected and what is actually occurring and then provide alerts 130 as appropriate for this. The overlay 100 could also include open loop information as well. The user could set the system 10 to tie in with CareLink® information and display charts 107 and other background information, as described by way of example in U.S. patent application Ser. No. 12/643,524 (Attorney Docket No. P0035643.06), filed Dec. 21, 2009, entitled DIABETES THERAPY MANAGEMENT SYSTEM, incorporated by reference in its entirety herein. All of the foregoing diabetes management information data values can be shown by the overlay 100 on a continuous, real-time basis.

As shown in FIGS. 1-3 and 6-9, embodiments of the system 10 can include an alert 130. The alert 130 can change the size of the overlay 100, expand the information content provided in the overlay 100, provide an alert sound 112, provide an alert message 104, provide an alert image 110, freeze the underlying display content 40, provide a change in color of the overlay 100 or underlying display content 40, or any combination thereof. As a non-limiting example, the size of the overlay 100 in relation to the underlying display content 40 can increase or decrease based on the importance of the alert 130, the duration of the alert 130, or the alert level of the information in the overlay 100. In some embodiments, the alert 130 can be within the boundary of the overlay 100 on the display screen 20, outside the boundary of the overlay 100 on the display screen 20, or can extend across the display screen 20, both within and outside the boundary of the overlay 100. In alternative embodiments, the alert 130 can be provided on a remote device 26. The remote device 26 can include, as non-limiting examples, glucose sensors 150, infusion devices 160, smart phones, tablets, computers or the like.

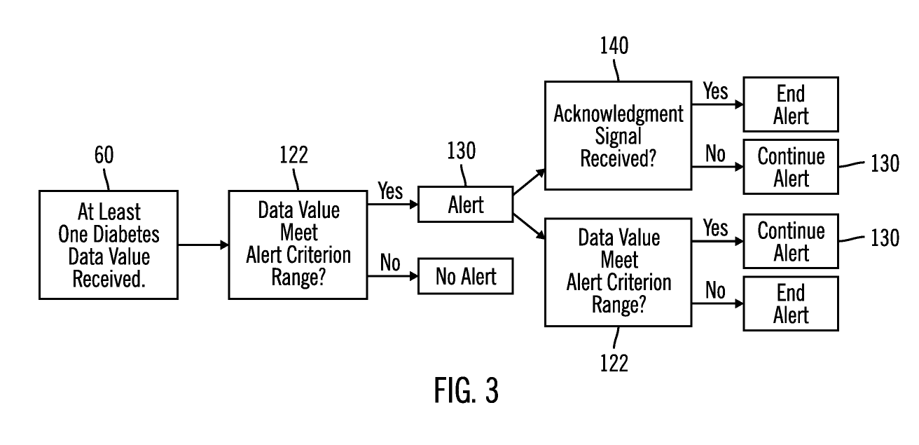

Embodiments of the system 10 can include a memory 120 having an alert criterion range 122 corresponding to one or more stored diabetes data values. As illustrated by way of an exemplary flowchart in FIG. 3, the processor 90 can compare a received diabetes data value 60 with the alert criterion range 122 and generate an alert 130 upon receiving at least one diabetes data value 60 within the alert criterion range 122. In an alternative embodiment, the alert criterion range 122 can correspond to a stored range of expected data values and generate an alert 130 upon receiving a diabetes data value 60 outside the expected data value range. The processor 90 can provide the alert 130 to the display screen 20 or a remote device 26 via an alert signal 132. The processor 90 can terminate or end the alert 130 upon receiving at least one diabetes data value 60 outside the alert criterion range 122 (or, in an alternative embodiment, receiving a diabetes data value 60 within the expected data value range), or by receiving an acknowledgment signal 140 entered by the user. The acknowledgment signal 140 may be manually entered by the user via any device included in the system 10 adapted to provide the signal to the processor 90. For example, the user can enter an acknowledgment signal 140 via a remote control 180, the device having the display screen 20, including a touch-screen programming screen, a remote device 26, or another device that can be included in the system 10 such as the insulin infusion device 160. Alternatively, the alert 130 can end when the processor 90 receives a glucose data value 70 or insulin data value 80 that corrects the condition that caused the alert 130.

In particular embodiments, the alert 130, like the overlay 100, can also provide arrows 114 and flashing 105 or scrolling 106 messages to alert 130 the user of a specific condition. As non-limiting examples, the overlay can include multiple arrows 114 pointing up or down, and can also increase or decrease the number of arrows 114 on display in accordance with the trending of the data values 60. All or portions of the information in the overlay 100 and/or alerts 130 can flash, move in any direction, or change color. The information is typically overlaid above the underlying display content 40 but can be placed and/or scrolled anywhere within the display. The overlay 100 and/or alert 130 can change position on the display screen 20 over time to draw attention to the information over time if there is no acknowledgment response from the user, or just to draw the eye. In some embodiments, the overlay 100 and/or alert 130 could be used with intelligence algorithms such that the overlay 100, a portion of the overlay 100, and/or alerts 130 can move to positions that obscure the least amount of picture or content within the underlying display content 40. In other embodiments, overlay 100 and/or alert 130 can change tones, change volume, or can communicate with an emergency contact.

In further embodiments, the alerts 130 can be scrolled across the screen 20 and may be in various colors and/or patterns to denote importance and to differentiate the alerts 130. The use of sound 112 may also be included with the alerts 130. For instance, the alert 130 could begin only with a visual effect and subsequently add sound 112 if there is no change in the alert condition or no corrective action occurs to end the alert 130. Alternatively, the alert 130 could start with a sound 112. In some embodiments where an alarm must be acknowledged, audible alarms associated with the overlay 100 may increase in volume while automatically decreasing the volume of the audio associated with the underlying display content 40.

Embodiments of the system 10 can include the capability to send the alert 130 to multiple sources. For example, the processor 90 of the system 10 can provide the alert 130 using an alert signal 132 to a different location, service, or remote device 26 adapted to receive and provide the alert 130. The processor 90 might prompt the user to request that an alert 130 be forwarded to the different location, device or service. Alternatively, if no response to an alert 130 is provided, the processor 90 could automatically escalate the alert 130 and send messages or alerts 130 to other locations or remote devices 26 such as one or more emergency numbers, paid monitoring services, smart phones, tablets, computers or the like. In alternative embodiments, a network can be set up to simultaneously display the overlay 100 or alert 130 across multiple devices.

As illustrated in FIGS. 1 and 2, embodiments of the system 10 can include a user-interactive remote control 180. In embodiments utilizing a remote control 180, the processor 90 can be adapted to accept and execute program instructions 182 from the remote. The remote can be a discrete component 24 of the system 10 and the system 10 can include a programming screen 190 on the display screen 20. In certain embodiments, the remote can include a touch screen or the programming screen 190 can be a touch-screen on the display screen 20 or other device of the system 10.

In some embodiments, a user of the system 10 may manually enter commands with the remote control 180 to control the devices of the system 10, including the glucose sensor 150 and insulin infusion device 160. The glucose sensor 150 can be adapted to receive and process a first communication 200 transmitted from the remote control 180 via the processor 90. Likewise, in further embodiments, the insulin infusion device 160 can be adapted to receive and process a second communication 210 transmitted from the remote control 180 via the processor 90.

In alternative embodiments, the system 10 is automated and does not require user intervention. Through a signal feedback system, in response to glucose data values 70 and insulin data values 80 received by the processor 90, the processor 90 can transmit a first communication 230 to control the glucose sensor 150 adapted to receive the first communication 230, and a second communication 240 to control the insulin infusion device 160 adapted to receive the second communication 240. The processor 90 can have one or more transmitters 92 and/or receivers 94 to transmit and/or receive instructions from the devices of the system 10.

In further embodiments, the user could use the remote control 180, programming screen 190, and/or another device in the system 10 to configure settings of the overlay 100. Thus, the overlay 100 may be interacted with and not be strictly a display of information. As discussed above, a patient, caregiver, parent or user can interact with the system 10 and respond to alerts 130 of the system 10. The user could set the system 10 to tie in with CareLink® information and display charts 107 and other background information, as described by way of example in U.S. patent application Ser. No. 12/643,524 (Attorney Docket No. P0035643.06), filed Dec. 21, 2009, entitled DIABETES THERAPY MANAGEMENT SYSTEM, incorporated by reference in its entirety herein. Also, the user could set up functions of the processor 90, such as the screensaver mode on a display screen 20 of a computer to activate a screensaver and/or a small display of the overlay 100 on the desktop when the computer is not in saver mode. Alternatively, the overlay 100 could be programmed to always remain on top.

In some embodiments, the source providing the data values to the processor 90 can include an identification signal 260. The processor 90 may differentiate different sources of the data values and display one or more of several overlays 100 corresponding to different devices. This embodiment may be particularly useful with families having multiple individuals using glucose sensors 150 and/or infusion devices 160. Accordingly, multiple glucose sensors 150 and infusion devices 160 can be utilized in the system 10 to send and display diabetes data values 60 simultaneously in the overlay 100. The identification signal 260 can also be an option for the user to designate that he or she is in front of the display screen 20. The system 10 may be programmed to display the overlay 100 only when the source of the identification signal 260 or the signal providing the diabetes data values 60 is within the vicinity of the display screen 20.

In another embodiment illustrated in FIG. 2, the system 10 can include a motion detector 220 adapted to receive motion commands. The system 10 can detect motion and/or accept motion commands (i.e., like Wii® or Kinect®) from the user to program, interact, configure or respond to data and/or alerts 130 on the overlay 100. The system 10 may detect that the user's glucose sensor 150 and/or infusion device 160 is within viewing range of the display screen 20 via the identification signal 260 and/or the motion detector 220. The system 10 may be programmed to display the overlay 100 only when the source of the identification signal 260 or the signal providing the diabetes data values 60 is within the vicinity of the display screen 20 or motion detector 220. In alternative embodiments, the system 10 may provide an alert 130 when the source of the identification signal 260 or the signal providing the diabetes data values 60 is out of range from the system 10.

All communications discussed herein can be transmitted by wired or wireless signal. Wireless technology can include Wi-Fi, Bluetooth, ZigBee, along with cellular communication standards such as but not limited to CDMA and GSM. Other communications include, but are not limited to IR and/or optical communication methods.

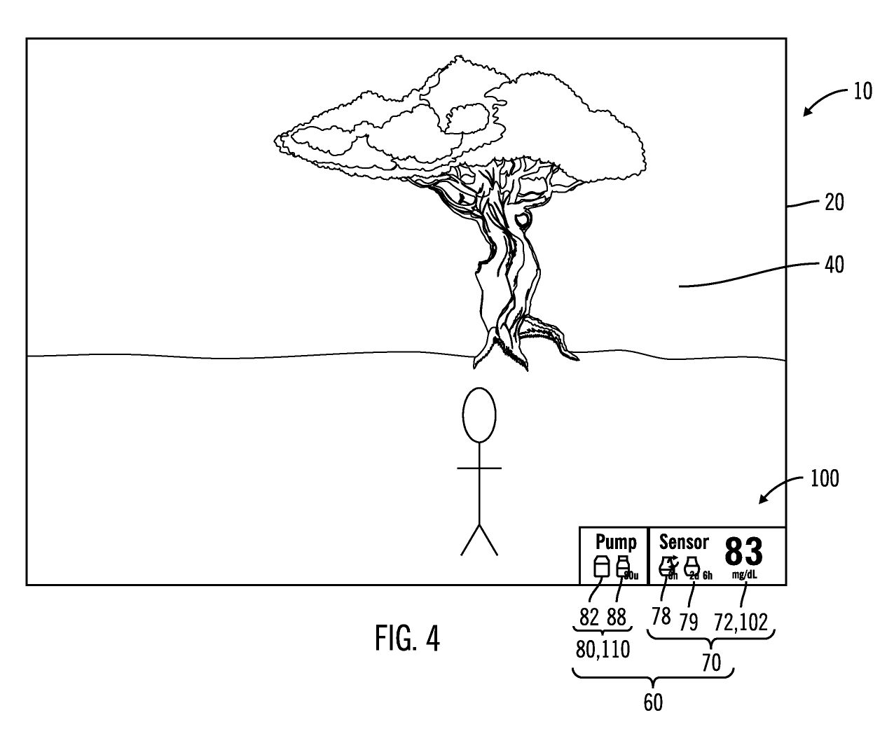

Turning now to further examples of embodiments of the overlay 100, FIGS. 4-9 are exemplary screenshots illustrating embodiments of overlaying real-time information from a glucose sensor 150 and insulin infusion device 160, along with real-time information regarding the status of each the glucose sensor 150 and insulin infusion device 160.

Generally in FIGS. 4-9, embodiments of the overlay 100 are displayed in the lower right corner of a display 20. In alternative embodiments, the overlay 100 may be in other locations such as a different corner or the center of the display 20. In some embodiments, the overlay 100 can move to different positions of the display 20. In alternative embodiments, portions of the overlay 100 can move, flash, scroll, increase or decrease in size, change color, and/or modify audio or visual effects. In the embodiment shown in FIGS. 4-9, insulin data values 80 including an insulin infusion device power supply indicator 82 and an insulin reservoir supply indicator 88. In these figures, the insulin infusion device power supply indicator 82 is an image 110 of a battery that is filled with a color when at 100% power and empties, lowering the color level in the battery, as the power is depleted. The power supply indicator could include a numeric value 102 or percentage of power supply remaining in the battery or both a numeric value 102 and image 110 of the battery. The insulin reservoir supply indicator 88 is similarly an image 110 of a reservoir that is filled with a color at 100%, where the color level of the reservoir lowers as the insulin supply is depleted. The insulin reservoir supply indicator 88 can also, but need not, include a numeric value 102 of the number of units of insulin remaining as shown in FIGS. 4-9.

In other embodiments, the insulin data values 80 can include insulin infusion parameters such as an insulin basal rate or an insulin bolus. The insulin data values 80 can be shown on the overlay 100 as text 104 or numeric values 102. In other embodiments, charts 107 and/or graphs 108 can show insulin data value 80 trends over a specified time period. The overlay 100 can comprise a combination of any visual and audio features as described and shown herein.

In embodiments shown in FIGS. 4-9, glucose data values 70 shown in the overlay 100 include glucose sensor data values such as glucose sensor calibration indicators 78 and glucose sensor power supply indicators 79. In embodiments having a glucose sensor calibration indicator 78, the overlay 100 can display a glucose sensor image 110 and a numeric value 102 that indicates the amount of time remaining until the glucose sensor 150 requires calibration. In embodiments showing glucose sensor power supply indicators, the overlays 100 include images 110 and numeric values 102 of the amount of time remaining prior to the life of the glucose sensor 150 expiring. In the alternative, the overlay 100 may display the amount of time the glucose sensor 150 has been in use. In one embodiment, the glucose sensor image 110 is filled with color and empties, or lowers the color level from the top of the sensor image 110 to the bottom of the sensor image 110, to indicate the glucose sensor 150 life remaining. In another embodiment, as the glucose sensor 150 is used, the glucose sensor image 110 can fill with color from the bottom of the sensor to the top of the sensor to indicate the length of use. In some embodiments, numeric values 102 can display the amount of time sensor has life remaining and/or the amount of time the sensor has been used in days, hours, or different increments of time. The numeric values 102 can accompany or replace the sensor images 110.

Further embodiments show a real-time glucose data value 70 in each of FIGS. 4-9 as a larger numeric value 102 displayed on the right side of the overlay 100, in milligrams per deciliter. The numeric value 102 can utilize a different measurement system 10 and be shown in any size or color and can flash, scroll, or otherwise move about the display 20.

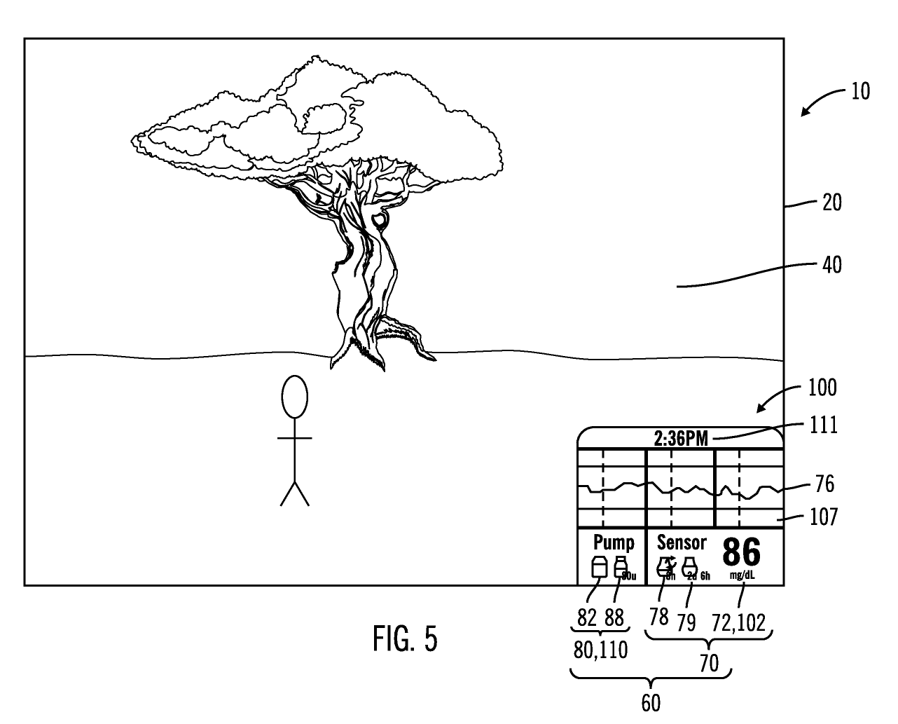

FIGS. 5, 6 and 8 also show embodiments with a graphical trend of glucose data values 70 over a specified time period. The time period increments can be adjusted by the user or set via the processor 90. All glucose data values 70 described herein can be transmitted from a glucose sensor 150 to both an infusion pump and additional receivers such as the processor 90 of the system 10 via a transmitter 152 coupled to the glucose sensor 150.

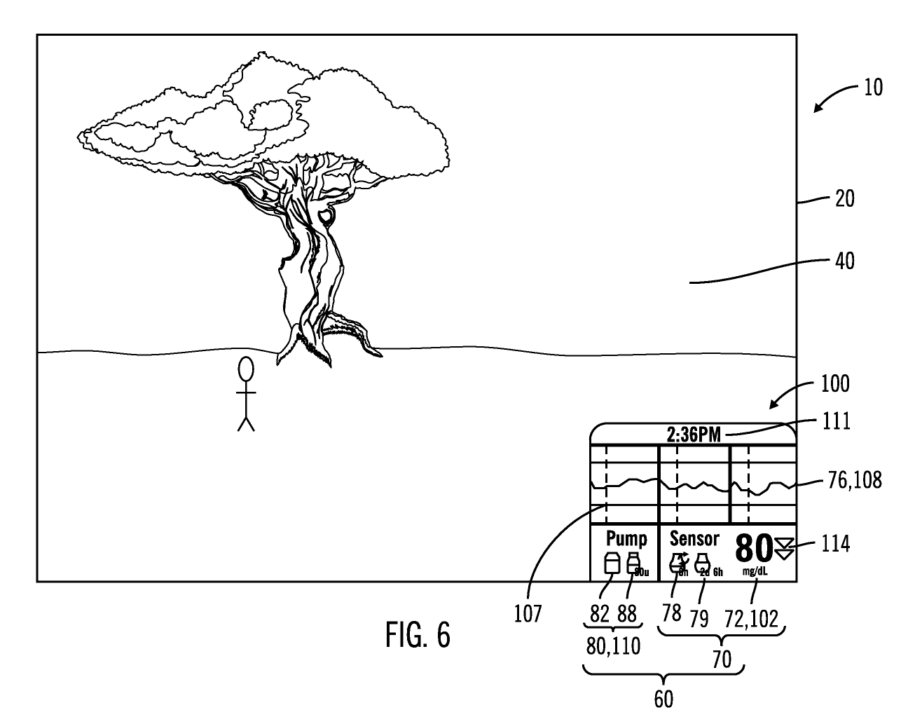

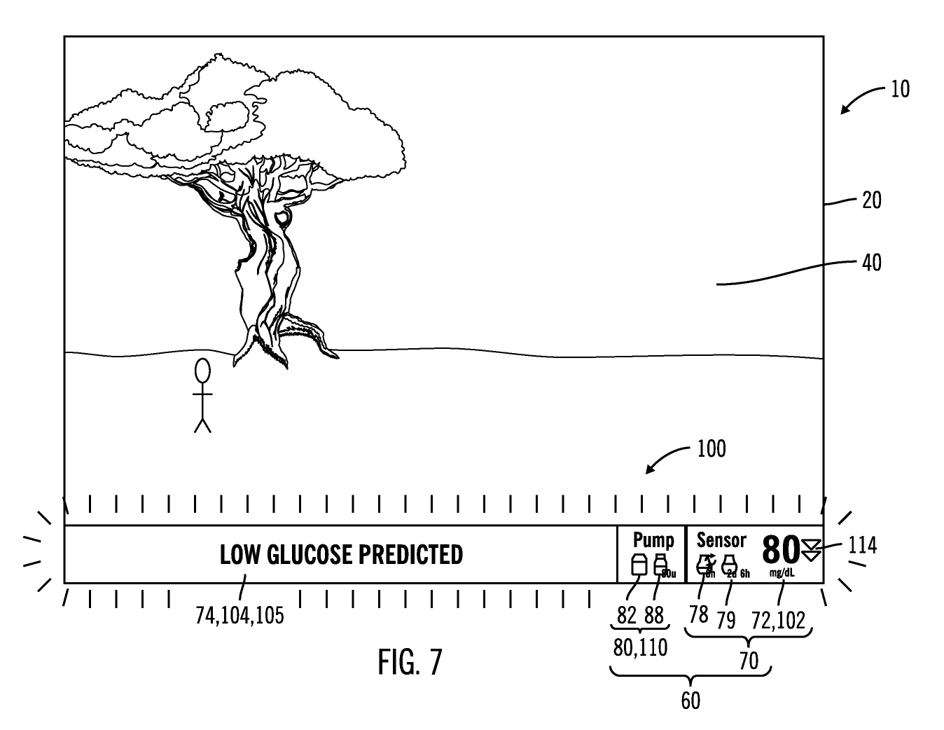

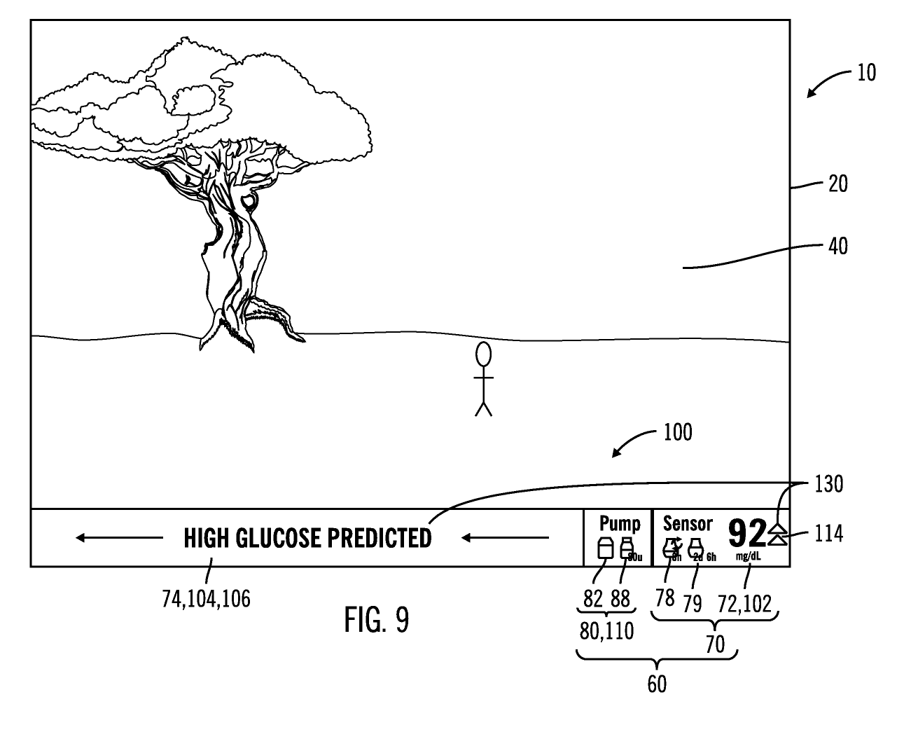

FIGS. 6-9 show embodiments of overlays 100 and/or alerts 130 utilizing messaging and arrow 114 features. In some embodiments, the overlay can include multiple arrows 114 pointing up or down, and can also increase or decrease the number and size of arrows 114 on display in accordance with the trending of the data values 60. The text 104 provided in the overlay 100 can be in the form of a flashing message 105 or a scrolling message 106. FIG. 7 illustrates one embodiment of a flashing message 105. FIG. 9 illustrates one embodiment of a scrolling message 106 that scrolls to the left. The message 104 may scroll up, down, to the right or at any angle across the display 20. The text 104 of the overlay 100 can be in shown in any location on the display 20 and can move locations within the display 20. All or portions of the information in the overlay 100 and/or alerts 130 can flash or change color.

FIG. 6 illustrates a situation where downward trending of blood glucose levels 76 has triggered an alert 130 indicating there is a risk of hypoglycemia. As a non-limiting example, the numeric value 102 of the blood glucose level 72 can change in color and an arrow 114 pointing down to indicate decreasing blood glucose levels is shown. As illustrated in FIG. 7, additional overlay information 100 can be displayed when low glucose is predicted. The additional information can be in the form of an alert 130 having extended overlay 100 that extends across the display screen 20 and further includes a message that scrolls across the display screen 20. While the extended overlay 100 in FIG. 7 is shown extending horizontally across the display screen 20, other embodiments may use different orientations of the text 104, occupy different areas of the display screen 20, and use different colors. In an effort to get the attention of viewers, the extended overlay 100 may further periodically flash or flash in different colors. In other embodiments, the extended overlay 100 may increase in size and obtrusiveness over the underlying display content 40 concurrently displayed on the display screen 20 until an acknowledgment signal or command 140 is received by the infusion device 160 or processor 90 of the system 10. In other embodiments the underlying content below the overlay 100 may be frozen in an effort to capture the attention of viewers.

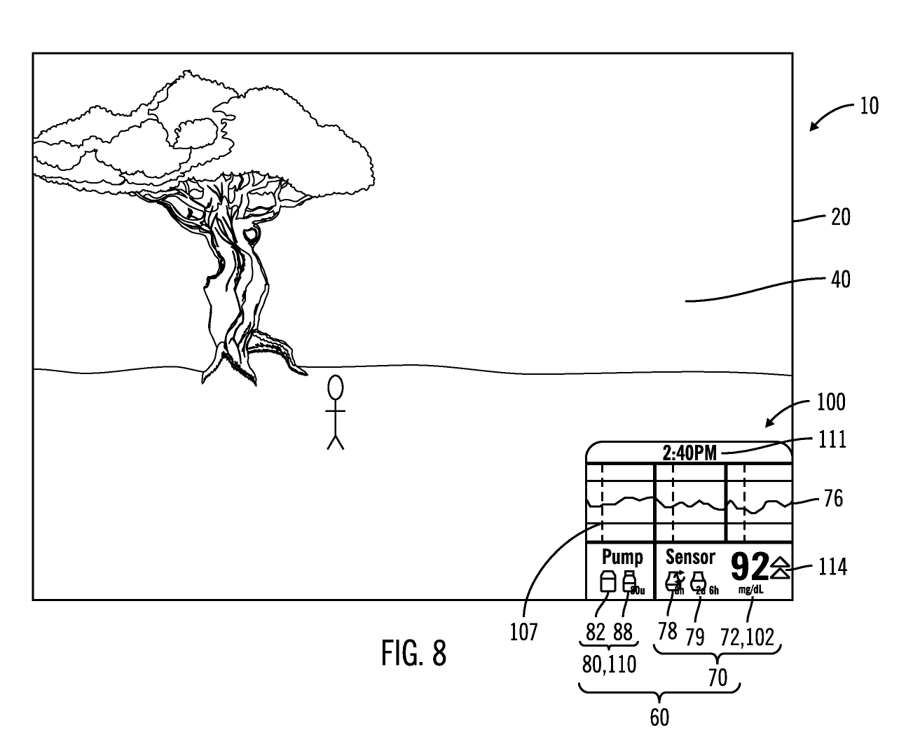

FIGS. 8 and 9 are exemplary illustrations of screenshots of embodiments where hyperglycemia is detected via the glucose sensor 150. FIG. 8 includes arrows 114 to the right of the glucose data values 70 that indicate the trending upward direction of glucose values detected by the sensor. In FIG. 9, a message “HIGH GLUCOSE PREDICTED” is scrolled through the extended overlay 100 while the original overlay 100 area displays pump status, sensor status, and glucose values detected by the sensor.

Though the combinations of a numeric data values 102, text 104, charts 107 or graphs 108, and images 110 shown in FIGS. 4-9 may be overlayed exactly as shown in the figures, any other suitable combination may be included in the overlay 100. A time stamp 111 may be included in any location of the overlay 100, and is shown by way of example in FIGS. 5, 6, and 8. In other embodiments the overlay 100 may be displayed in any other area of the screen and may further be resized. All of the features of the overlay 100 described herein, including color and sound 112, can be utilized alone or in combination.

Methods for overlaying glucose information on a display are also described herein by way of the embodiments described above. An example method can generally include: providing a display screen; receiving an input display signal for providing an underlying display content on the display screen; receiving an overlay signal including a diabetes data value, the diabetes data value at least one of a glucose data value and an insulin data value; providing a processor for generating an overlay based the at least one diabetes data value and superimposing the overlay over the underlying display content on the display screen. The methods can have fewer or additional steps to encompass all embodiments of the overlays and systems described herein, and need not be performed in any particular order.

The following patent applications, as identified by serial number, are incorporated in their entirety by reference herein: U.S. patent application Ser. No. 09/409,014 (Attorney Docket No. PF0000306.02), filed Sep. 29, 1999, entitled COMMUNICATION STATION AND SOFTWARE FOR INTERFACING WITH AN INFUSION PUMP, ANALYTE MONITOR, ANALYTE METER, OR THE LIKE; U.S. patent application Ser. No. 09/487,423 (Attorney Docket No. PF0000383.00), filed Jan. 20, 2000, entitled HANDHELD PERSONAL DATA ASSISTANT (PDA) WITH A MEDICAL DEVICE AND METHOD OF USING THE SAME; U.S. patent application Ser. No. 10/335,256 (Attorney Docket No. PF0001003.01), filed Dec. 31, 2002, entitled RELAY DEVICE FOR TRANSFERRING INFORMATION BETWEEN A SENSOR SYSTEM AND A FLUID DELIVERY SYSTEM; U.S. patent application Ser. No. 10/750,080 (Attorney Docket No. PF0001074.00), filed Dec. 31, 2003, entitled SYSTEM FOR MONITORING PHYSIOLOGICAL CHARACTERISTICS; U.S. patent application Ser. No. 10/860,114 (Attorney Docket No. PF0001127.00), filed Jun. 3, 3004, entitled SYSTEM FOR MONITORING PHYSIOLOGICAL CHARACTERISTICS; U.S. patent application Ser. No. 10/913,149 (Attorney Docket No. PF0001137.00), filed Aug. 6, 2004, entitled MEDICAL DATA MANAGEMENT SYSTEM AND PROCESS; U.S. patent application Ser. No. 11/225,359 (Attorney Docket No. P0022339.00), filed Sep. 13, 2005, entitled MODULAR EXTERNAL INFUSION DEVICE; U.S. patent application Ser. No. 11/172,492 (Attorney Docket No. P0023025.00), filed Jun. 29, 2005, entitled FLEXIBLE GLUCOSE ANALYSIS USING VARYING TIME REPORT DELTAS AND CONFIGURABLE GLUCOSE TARGET RANGES; U.S. patent application Ser. No. 11/413,268 (Attorney Docket No. P0025009.00), filed Apr. 28, 2006, entitled MONITOR DEVICES FOR NETWORKED FLUID INFUSION SYSTEMS; U.S. patent application Ser. No. 11/931,363 (Attorney Docket No. P0027630.01), filed Oct. 31, 2007, entitled SYSTEMS AND METHODS FOR DIABETES MANAGEMENT USING CONSUMER ELECTRONIC DEVICES; U.S. patent application Ser. No. 12/343,875 (Attorney Docket No. P0034275.00), filed Dec. 24, 2008, entitled PATTERN RECOGNITION AND FILTERING IN A THERAPY MANAGEMENT SYSTEM; and U.S. patent application Ser. No. 12/643,524 (Attorney Docket No. P0035643.06), filed Dec. 21, 2009, entitled DIABETES THERAPY MANAGEMENT SYSTEM.

Optional embodiments may combine elements of the overlay, system, and/or method of display in different ways. While the description above refers to particular embodiments of the present invention, it will be understood that many modifications can be made without departing from the spirit thereof. The accompanying claims are intended to cover such modifications as would fall with the true scope and spirit of the present invention.

The presently disclosed embodiments are therefore to be considered in all respects as illustrative and not restrictive, the scope of the invention being indicated by the appended claims, rather than the foregoing description, and all changes within the meaning and range of equivalency of the claims are therefore intended to be embodied therein.

What is claimed is:

1. A system for overlaying diabetes management information on a display comprising:
a display screen having an underlying display content;
a diabetes data value, wherein the diabetes data value is at least one of a glucose data value and an insulin data value; and
a processor for generating an overlay including the diabetes data value superimposed over the underlying display content on the display screen.

2. The system of claim 1, wherein the diabetes data value is continuous and includes at least one real-time data value.

3. The system of claim 1, wherein the glucose data value is at least one of a blood glucose level, a predicted blood glucose level, a plurality of blood glucose levels over a specified period of time, a glucose sensor calibration indicator, and a glucose sensor power supply indicator.

4. The system of claim 1, the overlay further including at least one of a numeric value, a text, a chart, a graph, an image, and a sound.

5. The system of claim 4, the overlay image further including an arrow pointing up to indicate an increased blood glucose level.

6. The system of claim 4, the overlay image further including an arrow pointing down to indicate a decreased blood glucose level.

7. The system of claim 4, the text including at least one of a flashing message and a scrolling message.

8. The system of claim 1, wherein the insulin data value includes at least one of an insulin infusion device power supply indicator, an insulin basal rate, an insulin bolus, and an insulin reservoir supply indicator.

9. The system of claim 1, wherein the processor analyzes the underlying display content and adjusts the overlay in relation to the underlying display content.

10. The system of claim 9, wherein the processor includes a screensaver function to display the overlay when the underlying display content is inactive.

11. The system of claim 1, the system further including a memory having an alert criterion range corresponding to one or more stored diabetes data values.

12. The system of claim 11, wherein the processor compares at least one diabetes data value with the alert criterion range, generates an alert upon receiving at least one diabetes data value within the alert criterion range, and provides the alert to the display screen.

13. The system of claim 12, wherein the alert is at least one of changing the size of the overlay in relation to the underlying display content, expanding the diabetes management information in the overlay, providing an alert sound, providing an alert message, providing an alert image, freezing the underlying display content, changing a color of the overlay, and changing a color of the underlying display content.

14. The system of claim 12, wherein the processor ends the alert upon receiving at least one of an acknowledgment signal and a diabetes data value outside the alert criterion range.

15. The system of claim 12, wherein the processor provides an alert signal including the alert to a remote device adapted to receive and provide the alert.

16. The system of claim 1, the system further including a glucose sensor and glucose sensor transmitter, wherein the glucose sensor transmitter is adapted to transmit the glucose data value to the processor.

17. The system of claim 1, the system further including an insulin infusion device and insulin infusion device transmitter, wherein the insulin infusion device transmitter is adapted to transmit the insulin data value to the processor.

18. The system of claim 1, the system further including an insulin reservoir.

19. The system of claim 1, wherein the display screen is located on a first object and the processor is located in the first object.

20. The system of claim 1, wherein the display screen is located on a first object and the processor is located in an external device in communication with the first object.

21. The system of claim 20, wherein the external device comprises a console.

22. The system of claim 1, the system further including a remote control.

23. The system of claim 22, wherein the processor is adapted to receive and process instructions transmitted from the remote control.

24. The system of claim 22, wherein the overlay includes a programming screen.

25. The system of claim 22, wherein the glucose sensor is adapted to receive and process a first communication transmitted from the remote control via the processor.

26. The system of claim 22, wherein the insulin infusion device is adapted to receive and process a second communication transmitted from the remote control via the processor.

27. The system of claim 1, the system further including a motion detector adapted to receive motion commands.

28. The system of claim 1, wherein the overlay further comprises an analyte reading.

29. The system of claim 1, the system further including an input display signal for providing the underlying display content on the display screen.

30. The system of claim 1, the system further including an overlay signal including the diabetes data value, wherein the processor is adapted to provide the overlay signal to the display screen.

31. A method for overlaying glucose information on a display comprising the steps of:
providing a display screen having an underlying display content on the display screen;
receiving at least one diabetes data value, wherein the at least one diabetes data value is at least one of a glucose data value and an insulin data value;
providing a processor for generating an overlay based the at least one diabetes data value; and
superimposing the overlay over the underlying display content on the display screen.

32. An overlay for displaying diabetes management information on a display comprising:
a display screen;
an underlying display content on the display screen;
a diabetes data value, wherein the diabetes data value is at least one of a glucose data value and an insulin data value;
whereby an overlay is formed by the at least one diabetes data value superimposed over the underlying display content on the display screen.
